(19) United States
(12) Patent Application Publication
(10) Pub. No.: US 2026/0260734 A1
(43) Pub. Date: Sep. 3, 2026

(54) DEEP LEARNING ENABLED B0 MAPPING BEYOND THE BRAIN

(71) Applicant: Siemens Healthineers AG, Forchheim (DE)

(72) Inventors: Cornelius Eichner, Fürth (DE); Shihan Qiu, Plainsboro, NJ (US); Radu Miron, Iasi (RO); Yahang Li, Plainsboro, NJ (US); Nirmal Janardhanan, Alpharetta, GA (US); Laszlo Lazar, Gheorgheni (RO); Bryan Clifford, Belmont, MA (US); Mahmoud Mostapha, Princeton, NJ (US); Omar Darwish, London (GB); Thorsten Feiweier, Poxdorf (DE); Mariappan S. Nadar, Plainsboro, NJ (US)

(21) Appl. No.: 19/269,587

(22) Filed: Jul. 15, 2025

Related U.S. Application Data
(60) Provisional application No. 63/764,587, filed on Feb. 28, 2025.

(30) Foreign Application Priority Data
Feb. 28, 2025 (EP) 25465513.7

(51) Int. Cl. G16H 30/40; G06N 3/0455; G06N 3/0464; G06T 5/80

(52) U.S. Cl. CPC G16H 30/40; G06N 3/0455; G06N 3/0464; G06T 5/80; G06T 2207/10088; G06T 2207/20081

(57) ABSTRACT

Systems and methods for distortion correction of medical imaging data. High quality reference data is acquired which is input into a specialized deep learning model for generalized distortion correction across various body regions (incl. brain, neck, pelvis). High quality reference data is acquired using two or more separate scans (i.e., blip-up, blip-down). By separating the reference scans from the actual scans, this allows for a reduction in the TE of the reference scans, thereby increasing the resulting SNR for B0 calculation. In addition, a novel architecture is provided for a DL model for DF estimation that uses Def-ConvFormer blocks that include a convolution modulation block with a deformable convolution.

### CROSS REFERENCE TO RELATED APPLICATIONS

This application claims priority to U.S. provisional application Ser. No. 63/764,587 filed Feb. 28, 2025, and European Patent Application EP25465513.7, filed Feb. 28, 2025, both of which are entirely incorporated by reference.

### FIELD

This disclosure relates to medical imaging.

### BACKGROUND

Magnetic resonance imaging, or MRI, is a noninvasive medical imaging test that can generate detailed images of almost every internal structure in the human body, including, for example, organs, bones, muscles, and blood vessels. Traditional MRI is slow due to the need for sequential data acquisition, often resulting in long scan times that can cause patient discomfort and motion artifacts. Echo-planar imaging (EPI) enables fast MRI acquisitions and has been widely adopted in diffusion-weighted imaging (DWI) and functional MRI (fMRI). However, due to off-resonant spins, EPI is prone to geometric distortions along the applied phase encoding (PE) directions. Geometric distortions result in spatially varying displacements (shifts) of voxel information in images. These distortions can significantly impair clinical evaluation and can result in significant costs, i.e., delayed diagnoses, repeat exams, hampered treatment decisions etc. Additionally, distorted images can lead to down-stream analysis pipeline errors, such as incorrect registration of e.g., functional, or diffusion-weighted imaging data to undistorted imaging data.

This problem has garnered significant research attention from the MR community. However, most methods have yet to be successfully deployed in clinical environments. Some approaches estimate the B0 displacement field (DF) and use it to correct the distorted image as a post-processing step. One may fit the B0 field map from the phase images of a separate multi-echo gradient echo (GRE) scan, but it requires extra scan time and usually operates at low resolution. The motion between the GRE scan and the EPI scans can also cause mismatch. Alternatively, one may estimate the displacement field from a pair of EPI scans with reversed phase-encoding (PE) direction (i.e., blip-up and blip-down scans). The extra scan time is shorter. However, the displacement field estimation is an ill-posed problem. Typical iterative solver-based methods are computationally expensive and require more time than is practical in most clinical environments. Furthermore, the typically employed approaches were developed in the context of neuroimaging and may produce subpar results outside the brain region. This is due to challenges, such as low signal-to-noise ratio (SNR) and physiological motion in those regions.

### SUMMARY

By way of introduction, the preferred embodiments described below include methods, systems, instructions, and/or computer readable media for distortion correction of medical imaging data.

In a first aspect, a system for deep learning (DL) based distortion correction is provided, the system comprising: a medical imaging scanner configured to acquire a reference data set comprised of two acquisitions including a first acquisition with blip-up (BU) and the other with blip-down (BD) phase encoding, and an acquisition dataset of a region of a patient; an image processing unit configured to estimate a displacement field based on the reference dataset using a deep learning model, wherein the deep learning model comprises an encoder-decoder architecture with one or more deformable convolutional modulation blocks; the image processing unit configured to correct the acquisition dataset using the estimated displacement field.

In a second aspect, a method for training a network for deep learning based distortion correction is provided, the method comprising: acquiring a training dataset comprising pairs of reference blip-up and blip-down acquisition data; iteratively training the network to estimate a displacement field for use in distortion correction of acquired image data for a patient when input a respective pair of reference blip-up and blip-down data, the network comprising an encoder-decoder architecture with one or more deformable convolutional modulation blocks, the training comprising minimizing a loss function comprising a mean square error (MSE) between two corrected images, a spatially weighted smoothness loss on the estimated displacement field, and optional supervised losses on the estimated displacement field and/or corrected images; and storing the trained network.

In a third aspect, a method for deep learning enabled B0 mapping is provided, the method comprising: acquiring, by a medical imaging scanner, a high quality BUDA dataset; acquiring, by the medical imaging scanner, an acquisition dataset of a region of a patient; estimating, using a deep learning model, a displacement field based on the high quality BUDA dataset, the deep learning model comprising encoder-decoder architecture with one or more deformable convolutional modulation blocks; and correcting the acquisition dataset using the displacement field.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale; emphasis instead being placed upon illustrating the principles of the embodiments. Moreover, in the figures, like reference numerals designate corresponding parts throughout the different views.

FIG. 1 depicts an example system for generalized distortion correction according to an embodiment.

FIG. 2 depicts an example fast B0 reference scan.

FIG. 3 depicts a high quality B0 reference scan according to an embodiment.

FIG. 4 depicts an example architecture for a deep learning model for displacement field estimation according to an embodiment.

FIG. 5 depicts an example method for training a deep learning model for displacement field estimation according to an embodiment.

FIG. 6 depicts an example of corrected images using different methods according to an embodiment.

FIG. 7 depicts an example method for applying a deep learning model for displacement field estimation according to an embodiment.

FIG. 8 depicts example distortion corrections according to an embodiment.

FIG. 9 depicts an example image processing unit according to an embodiment.

FIG. 10 depicts an example artificial neural network according to an embodiment.

FIG. 11 depicts an example convolutional neural network according to an embodiment.

FIG. 12 depicts an example U-net architecture according to an embodiment.

### DETAILED DESCRIPTION

Embodiments described herein provide systems and methods that acquire high quality reference data that is input into a specialized deep learning model for generalized distortion correction across various body regions (incl. brain, neck, pelvis). High quality reference data is acquired using a reference scan as two or more separate scans (i.e., blip-up, blip-down). By separating the reference scans from the actual scans, this allows for a reduction in the echo time (TE) of the reference scans, thereby increasing the resulting SNR for B0 calculation. In addition, a novel architecture is provided for a DL model for DF estimation that uses Def-ConvFormer blocks, a convolutional modulation block with deformable convolution, among other adaptions from a typical encoder-decoder architecture with skip connections. The DL model for displacement field estimation provides stable distortion correction performance across various anatomies and achieves significantly faster computation compared to iterative methods particularly when trained or implemented with the high quality reference data.

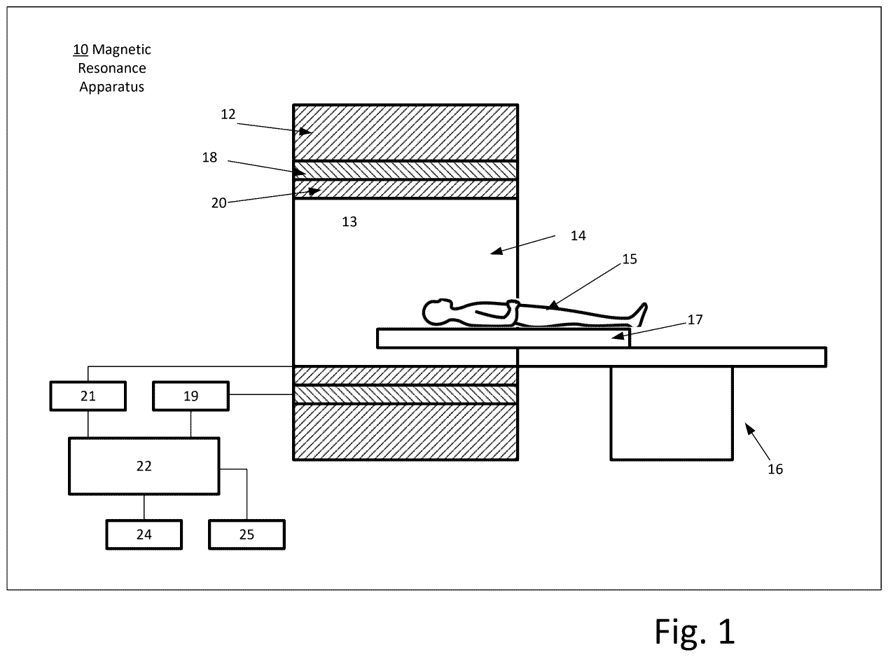

FIG. 1 depicts an example magnetic resonance apparatus 10 (medical imaging scanner) that may be used for generalized distortion correction. The magnetic resonance apparatus 10 includes a magnetic unit that includes a main magnet 12 for the generation of a main magnetic field 13. In addition, the magnetic resonance apparatus 10 includes a patient receiving area 14 for receiving a patient 15. The patient receiving area 14 may be cylindrical in design and cylindrically surrounded by the magnetic unit in a circumferential direction. Different designs of the patient receiving area 14 may be used. The patient 15 may be pushed into the patient receiving area 14 by a patient positioning device of the magnetic resonance apparatus 10. The patient positioning device includes a patient table 17 for this purpose that is configured to be movable within the patient receiving area 14.

The magnetic unit also includes a gradient coil unit 18 for the generation of gradient pulses that are used for location coding during imaging. The gradient coil unit 18 is controlled by a gradient control unit 19 of the magnetic resonance apparatus 10. The magnetic unit also includes a radio frequency antenna unit 20, that may be configured as a body coil permanently integrated into the magnetic resonance apparatus 10. The radio frequency antenna unit 20 is controlled by a radio frequency antenna control unit 21 of the magnetic resonance apparatus 10 and emits RF transmission pulses into an examination area during a magnetic resonance measurement, that is essentially formed by a patient 15 receiving area 14 of the magnetic resonance apparatus 10. As a result, atomic nuclei which are present in the main magnet field generated by the main magnet 12 are excited. Magnetic resonance signals are generated by relaxation of the excited atomic nuclei. The radio frequency antenna unit 20 is configured to receive magnetic resonance signals.

The magnetic resonance apparatus 10 includes a system control unit 22 for controlling the main magnet 12, the gradient control unit 19 and for controlling the radio frequency antenna control unit 21. The system control unit 22 controls the magnetic resonance apparatus 10, such as, for example, performing a magnetic resonance measurement. The system control unit 22 may be configured to execute a computer-implemented method for generating magnetic resonance images among other tasks.

In addition, the system control unit 22 includes an image processing unit 60, not shown here in more detail, for evaluating magnetic resonance signals that are recorded during a magnetic resonance examination. The image processing unit 60 includes a processor 62, memory 64, and interface 66. Furthermore, the magnetic resonance apparatus 10 may also include a user interface for a medical operator which is connected to the system control unit 22. Control information such as, for example imaging parameters, as well as reconstructed magnetic resonance images may be displayed on a display unit 24, for example on at least one monitor. Furthermore, the user interface has an input unit 25 by which information and/or parameters may be entered by the medical operator during a measurement process. The system control unit 22 is further configured to optimize the static field correction (SFC) reference data acquisition using a high quality reference scan comprised of two acquisitions: one with “blip-up” and the other with “blip-down” phase encoding, also referred to as the “blip-reversed reference dataset”. The system control unit 22 is further configured to apply a DL model for DF estimation to the blip-reversed reference dataset.

In an imaging procedure, a MRI acquisition involves placing a patient 15 in a strong magnetic field and applying controlled radiofrequency pulses and gradient magnetic fields. These gradients encode spatial information into the MRI signal, collected as frequency-domain data, known as k-space. Reconstruction transforms k-space data into anatomical images. In MRI, the B0 field refers to the main, static magnetic field produced by the scanner, which is a strong, constant magnetic field that aligns the protons in the patient's tissues, allowing for the detection of their signal during an MRI scan. During a scan, this field may suffer from various distortions. Embodiments described herein provide systems and methods that attempt to estimate and correct for these distortions using a reference scan and a deep learning model.

Echo Planar Imaging (EPI) is a fast MRI technique that acquires many k-space lines following a single radiofrequency (RF) excitation, enabling an entire two-dimensional k-space to be captured in one shot, as in single-shot EPI, or divided over only a few excitations, as in multi-shot EPI. Unlike conventional MRI sequences, which acquire one k-space line per excitation, EPI rapidly collects spatial frequency data (k-space) using a series of gradient echoes within one repetition time (TR). This is achieved by quickly switching the readout and phase-encoding gradients in a zigzag pattern, allowing for extremely rapid imaging. One drawback of EPI is that EPI is particularly sensitive to magnetic field inhomogeneities and susceptibility artifacts, often resulting in geometric distortions and signal reduction or pileups, especially near air-tissue interfaces. Uncorrected distortions compromise diagnostic accuracy, reduce reliability of quantitative measurements, impair multimodal image alignment, may reduce the accuracy of radiotherapy planning, and negatively affect surgical planning, clinical assessments, and advanced medical imaging analyses.

Various MRI distortion correction methods have been used to address signal intensity and geometric inaccuracies due to magnetic field inhomogeneities. In an example, the distortions may be corrected using a static field correction (SFC) method. In the basic SFC approach, a low resolution B0-map (which is proportional to the displacement field) is acquired by an additional scan (typically a multi-echo gradient echo scan) and is used to correct the distorted image via a conventional unwarping algorithm (e.g., image interpolation followed by an image intensity correction step, wherein the intensity correction typically utilizes the Jacobian of the displacement field). This basic SFC distortion correction method has two main drawbacks: (i) it requires additional time to acquire separate scan(s), used for displacement field estimation, and (ii) as the displacement field is estimated from a separate scan (typically at lower resolution and at temporally distant), the estimated DF may not be fully aligned with the imaging data and thereby lead to imperfect correction/unwarping.

A second approach to SFC is to acquire images with opposing PE directions (e.g., Up Down-Down Up, or Left Right-Right Left), here referred to as blip-up and blip-down acquisitions (BUDA) and to estimate the displacement field that minimizes the difference between two images using an iterative optimization technique. This approach has the advantage of estimating a field that is consistent with the measured imaging data. In this context several algorithms have been proposed such as the TOPUP algorithm (from FSL), HySCO, and TISAC, etc. However, the computational cost of these algorithms is prohibitively high, making them infeasible to deploy in a clinical setting. Furthermore, these algorithms have typically been optimized for the brain and may fail at low SNR, leading to limited applicability in other body regions.

Recently, deep learning (DL) approaches have also been proposed to accelerate the distortion correction process. However, these techniques are highly optimized for specific use cases e.g., acquisition protocol, contrast, sampling-rate, and anatomy, etc. This limits the clinical utility of such approaches, since most clinical environments cover a broad range of use cases and operating conditions. In addition, because the performance of iterative approaches outside the brain is often poor, it is difficult to generate distortion-corrected/mitigated images for body regions other than the brain which are suitable for use as targets in a supervised machine learning training framework. In particular, DL model inference outside the brain is currently subpar for two reasons: the SNR in the remaining body regions is too low for the model to perform distortion correction with iterative algorithms or another non-learning-based method, and as only brain data could be included in the training set, the models fail to generalize across anatomies.

Embodiments provide an integrated scheme to acquire a blip-reversed reference dataset, and a specialized DL model for generalized distortion correction across various body regions (incl. brain, neck, pelvis). The enhanced SNR in the blip-reversed reference dataset improves the precision and robustness of estimated B0 maps, yielding better accuracy of image corrections (relevant during inference and/or training). This enables robust application of distortion correction in various body regions. The integrated approach to acquire blip-reversed reference dataset may shorten the general examination durations, as no separate scan needs to be performed. This approach may also improve the robustness of the reference data against subject motion. In addition, the specialized DL model for generalized distortion correction is stable across various anatomies and is substantially faster than iterative methods. The specialized DL model inference is additionally more stable than iterative methods in specific regions of high distortion, where iterative methods may fail in fully correcting the distortions, but DL promotes data integrity.

The blip-reversed reference dataset may be acquired using the MRI system of FIG. 1 by using a dedicated reference scan with its own TE that is different from the TE used by the actual acquisition scan. The combined dataset of the blip-up and blip-down scans may also be referred to as the reference dataset.

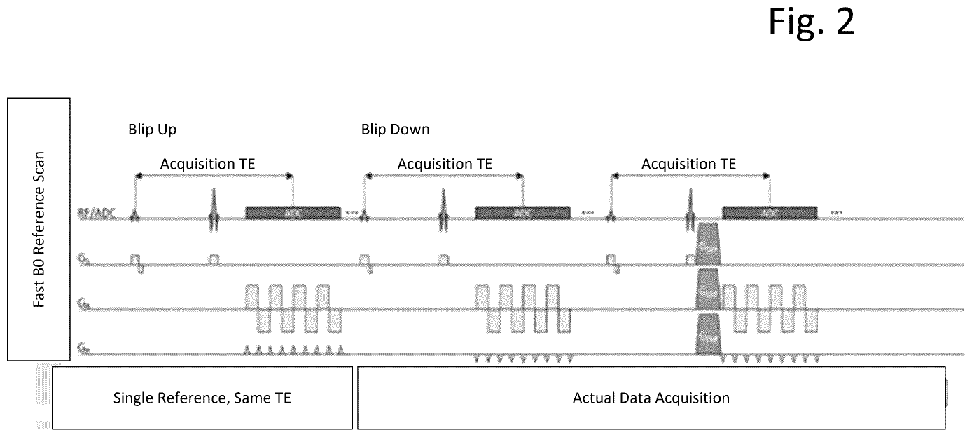

One way to perform a BUDA acquisition is by performing one additional acquisition of one EPI volume with a reversed PE direction. BUDA data may also be acquired as separate protocols outside the typical data acquisition. FIG. 2 depicts an example of a “fast” BUDA acquisition, also referred to as a fast BUDA B0 reference scan. The blip-up B0 reference scan is performed with the same TE as the actual data acquisition. As the remaining data acquisition is blip-down, the first scan is used as blip-up part of the BUDA acquisition. This approach provides a full BUDA dataset by only acquiring one additional blip-up dataset (Fast). In a DWI context, the BUDA reference dataset is typically acquired without diffusion weighting (to increase the available SNR), or with a low b-value (e.g., in abdominal imaging, where the suppression of fluid signal contributions is desirable). This dataset is then used to generate the displacement field and perform distortion correction by a one-dimensional nonlinear co-registration. While this fast strategy only requires one additional B0 volume, it restricts the available SNR to the one which is also used for data acquisition. In the brain, this approach typically generates sufficient SNR for B0 computation. Outside the brain, e.g., in the pelvis, SNR is generally too low to reliably generate a BUDA B0 field using such methods. Averaging may be employed to increase the SNR of the data. However, non-rigid physiological motion between the sets of reference data reduces the precision of the distortion fields and useability for ground truth generation.

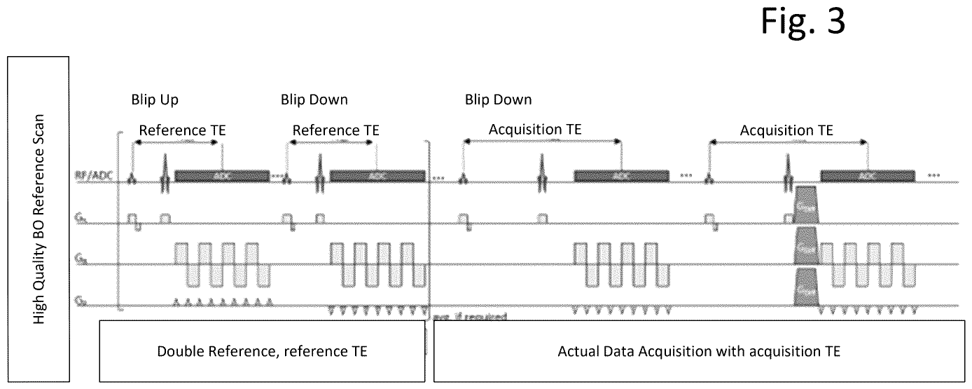

In an embodiment, the reference data acquisition is adapted as described in FIG. 3. FIG. 3 depicts a high-quality reference scan where the reference data is acquired as two or more separate scans (i.e., blip-up and blip-down) that are distinct from the actual acquisition scan. When acquiring both blip-up and blip-down images in the reference scan itself, the TE of the reference scan does not need to be matched with the actual data acquisition and can be shorter. In DWI, the reference dataset is typically acquired without diffusion weighting or with a low b-value, which allows substantial reductions of the underlying TE. This strategy increases the resulting SNR for B0 calculation and allows to measure reference data in low-SNR regions. The distortion characteristics of the high-quality reference scans may be the same as used in the imaging scans. For example, the same EPI readout module with identical sensitivity to B0 variations is used. However, since the sensitivity may be known a different EPI module may be used for the reference scan. This might be beneficial in two aspects, shaping the sensitivity of the reference scan and further increasing the SNR of the reference scan. If expected distortions are huge, one might consider using a smaller sensitivity of the reference scans in order to possibly improve precision. If expected distortions are small, one might consider using a higher sensitivity of the reference scans in order to possible improve precision. In addition, by reducing the spatial resolution of the reference scan, it may be possible to further boost SNR using larger voxels.

Additional averages of the reference scan may be included in an interleaved fashion between blip-up and blip-down. This approach may increase the reference acquisition time but will generate reference data of higher quality. Adapted multi-average acquisition may be performed in an interleaved fashion between PE directions (i.e., BU, BD, BU, BD, BU, BD vs. BU, BU, BU, BD, BD, BD). This acquisition strategy ensures minimized physiological differences (e.g., arising from possibly non-rigid motion) between PE directions. When averaging is employed to increase the SNR, the physiological motion is blurred out, while the susceptibility induced differences remain steady and can be reliably detected for distortion correction. Different averaging strategies may be used. For example, one evaluation method may include averaging of BU and BD, respectively, then calculating distortion maps based on the averages. This has the advantage of high SNR of the BU BD input images. Another evaluation method may include estimation of distortion maps for each pair of BU and BD reference scans, and then averaging of the distortion maps. This has the advantage of a good alignment of each pair. One might consider additional processing when combining the distortion maps (e.g., outlier removal, motion correction).

The blip-reversed reference dataset may be used by the image processing unit 60 of the MRI system 10 to estimate the displacement field and provide distortion correction, for example, using a specialized DL model for generalized distortion correction. If available, high-quality BUDA reference data may also be used to train the specialized DL model for generalized distortion correction.

A DL model can be trained to estimate the displacement field from the blip-up and blip-down images for SFC. Existing DL-based BUDA SFC approaches use a basic convolutional neural network (CNN) as the DL model, for example a U-Net with 3×3 convolutional kernels. Although effective in many image processing tasks, these conventional CNNs rely on fixed convolutional kernels which limits their ability to learn robust features for capturing the large local displacement in highly distorted BU BD image pairs. In EPI, the susceptibility-induced distortions vary spatially, and large distortions can be expected at regions with strong susceptibility artifacts. Therefore, network architectures with large and adaptive receptive fields are favorable.

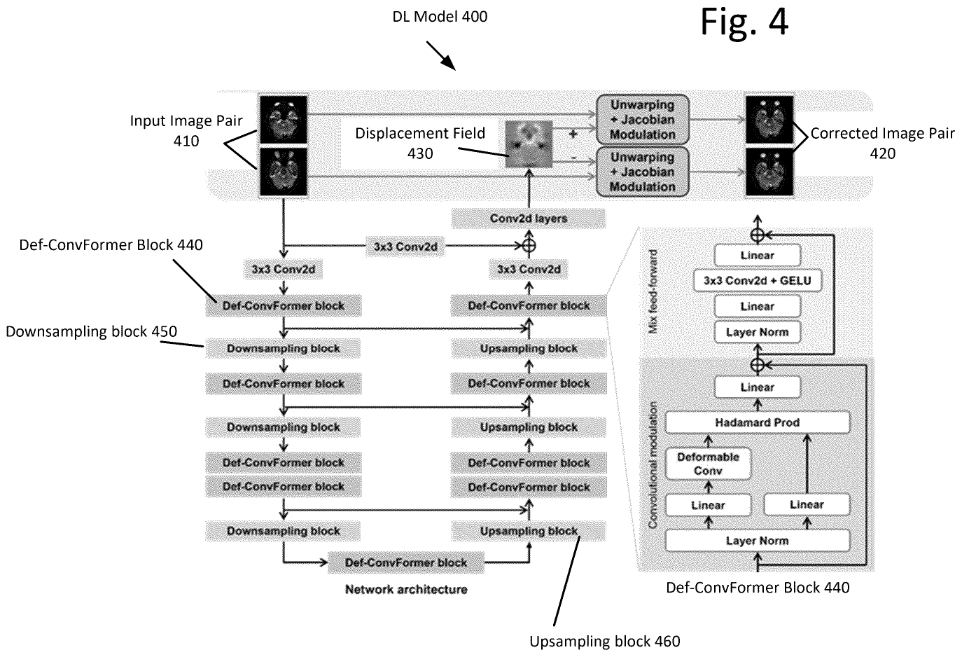

FIG. 4 depicts an example of the network architecture of the specialized DL model 400 for generalized distortion correction. The specialized DL model 400 for generalized distortion correction is similar to a U-Net as it uses an encoder-decoder architecture with skip connections. However, the architecture of the typical U-Net is adapted to use ConvFormer blocks, more specifically in Def-ConvFormer Blocks 440 as depicted in FIG. 4. In operation, the model 400 inputs an input image pair 410 into the encoder side of the network architecture that includes a plurality of Def-ConvFormer blocks 440 and down-sampling blocks 450. On the decoder side, the network includes a plurality of Def-ConvFormer blocks 440 and up-sampling blocks 460. A displacement field 430 is estimated which is then used to generate a corrected image pair 420. The displacement field 430 may be used to correct additional images of the patient 15.

A ConvFormer block is similar to a vision transformer that includes self-attention layers and feed forward layers but replaces the self-attention layer with a convolution modulation layer. By using a Hadamard product between the output of a large-kernel convolution and the value features, the convolution modulation layer mimics the longer-range dependencies in self-attention mechanism but considerably reduces computational cost. The computation of a convolution modulation layer is as follows:

$$A = D C o n {v}_{k \times k} ( {W}_{1} X ) ,$$

$$V = {W}_{2} X ,$$

$$Z = {W}_{3} ( A ⊙ V ) + X ,$$

where X is input of the layer, Z is output of the layer, and DConv_{k×k} is a depth-wise convolution with k×k kernel size. This architecture provides for each location to be correlated with all pixels within the k×k window centered at this location.

The large-kernel depth-wise convolution is further replaced in the convolution modulation layer with a deformable convolution. To perform a conventional 2D convolution, the network has to use a regular grid (R={(−1, −1), (−1,0), . . . , (1, 1)} for example in the case of a 3×3 kernel) over the input feature map to sample and then perform weighted summation of the sampled values:

$$y ( {p}_{0} ) = {\sum }_{{p}_{n} ∈ R} w ( {p}_{n} ) \cdot x ( {p}_{0} + {p}_{n} )$$

In deformable convolution, the regular grid is augmented with fractional offsets, leading to irregular sampling:

$$y ( {p}_{0} ) = {\sum }_{{p}_{n} ∈ R} w ( {p}_{n} ) \cdot x ( {P}_{0} + {P}_{n} + {Δ}_{P n} )$$

This learnable offsets for different locations enable the network to have adaptive receptive field size and can bring benefits in correcting spatially varying distortions in EPI. The final implementation for the renovated deformable convolutional modulation block is:

$$A = DeforConv ( {W}_{1} X ) ,$$

$$V = {W}_{2} X$$

$$Z = {W}_{3} ( A ⊙ V ) + X ,$$

Referring back to FIG. 4, the encoder architecture includes resolution levels with one or several deformable convolutional modulation blocks (the Def-ConvFormer Blocks 440). Besides the Def-ConvFormer Blocks 440, down-sampling blocks 450 are used to halve the current feature map's resolution. The decoder works in a symmetrical manner and includes, for each corresponding resolution level in the encoder, an up-sampling block 460. A residual connection to the input is made in the last resolution level. An 1×1 convolution is used to predict the displacement field 430. The model 400 is equipped with spatial transformer block (distortion correction block) for unwarping and Jacobian modulation for intensity correction. These blocks are applied after the prediction of the displacement field 430.

As described above with respect to FIG. 4, the network includes an encoder-decoder architecture built with Def-ConvFormer Blocks 440. In an embodiment, the network takes a reversed-PE image pair and outputs a displacement field 430, which is used to correct the distorted images through image unwarping and Jacobian modulation. Alternatively, the network may directly output corrected images or both the displacement field and the corrected images. Given a deformation field which maps original coordinates to new ones, unwarping seeks to compute the inverse map. This inverse is used to resample the deformed image back to its original geometry. Since the inverse deformation is typically not analytically available, it is approximated numerically. Once the inverse field is known, each pixel in the original image domain is filled by interpolating the intensity of the warped image at the corresponding deformed position. Jacobian modulation complements this process by correcting for intensity distortions introduced by the deformation. The Jacobian determinant quantifies local expansion or compression due to the transformation. To preserve physical or anatomical consistency, intensities are scaled by this determinant. Additional optional modules of the DL-based distortion correction framework include denoisers, which can be used to denoise the input reversed-PE images before displacement field estimation, and motion correction modules to correct for motion between the input image pair.

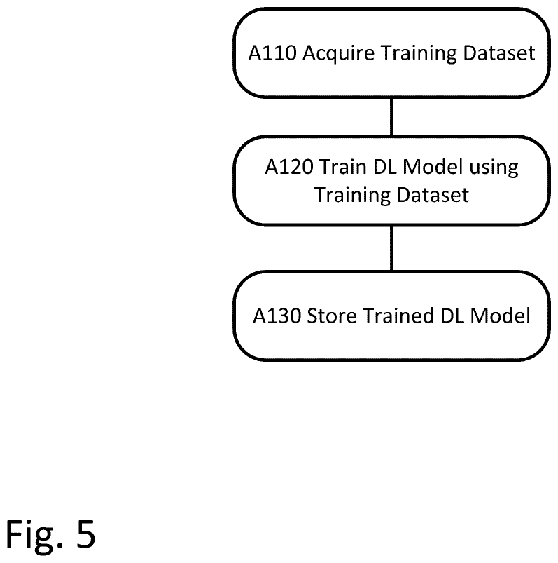

FIG. 5 depicts an example method for configuring/training the specialized DL model 400 for generalized distortion correction. The acts are performed by the system of FIGS. 1, 4, 9, other systems, a workstation, a computer, and/or a server. Additional, different, or fewer acts may be provided. The acts are performed in the order shown (e.g., top to bottom) or other orders. Certain acts may be omitted or changed depending on the results of the previous acts. The image processing unit 60 is configured to implement one or more machine learning networks that are stored in the memory. The image processing unit 60 or other computing unit is configured for configuring/training the network of the specialized DL model 400. In general, a trained machine learning network mimics cognitive functions that humans associate with other human minds. In particular, by training based on training data, the machine learning network is able to adapt to new circumstances and to detect and extrapolate patterns. Another term for “trained machine learning network” is “trained function”. In general, parameters of the machine learning network are adapted by means of training. Supervised training, semi-supervised training, unsupervised training, reinforcement learning and/or active learning may be used for training the network. Furthermore, representation learning (an alternative term is “feature learning”) may be used. In particular, the parameters of the machine learning networks may be adapted iteratively by several steps of training. The training attempts to minimize a cost function. The iterative results are used to adapt the network using backpropagation among other techniques. In an embodiment, the model 400 is trained end to end. Alternatively, individual components such as the network may be trained separately and then combined with other components.

At Act A110, a training dataset is acquired or provided. The dataset(s) used for training and evaluating the network include data for multiple organs with various EPI sequences and parameters. In an example, the training dataset is a diverse DL training dataset covering multiple anatomies, including the pelvis, head/neck, and brain. In an example, the images may be low b-value DWI or anatomical EPI images (T1-weighted, T2-weighted, T2*-weighted, etc.). The images can be based on single-shot EPI or multi-shot EPI sequences. Data splitting may be performed subject-wise within each dataset, resulting in scans for training/validation/testing.

In an embodiment, the training dataset is high-quality BUDA reference data, for example acquired as described in FIG. 3. Alternatively, a fast or conventional acquisition may be used to acquire the training data. The data may be labeled or not labeled. Labeling (i.e., reference correction) may be performed using existing algorithms.

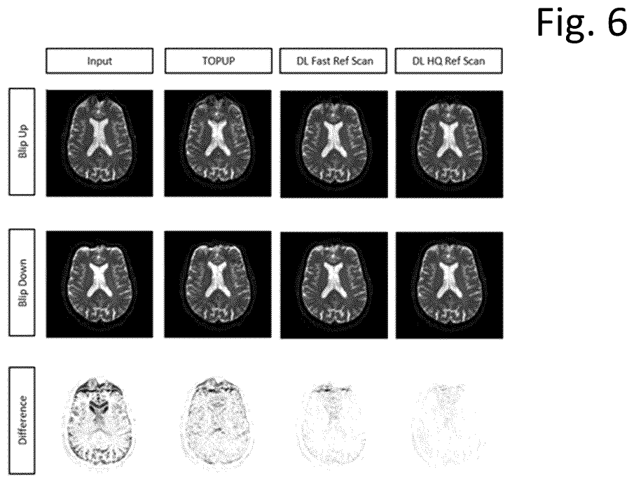

FIG. 6 depicts an example of different results from different methods and different training datasets. FIG. 6 depicts the BUDA input and output data, alongside their corresponding difference. The input data used to test the networks were not acquired with adapted acquisition (i.e., high-quality reference), but using conventional clinical parameters. Ideally, for perfect B0 correction, the difference image should contain only residual noise. It is visible from the examples that the different correction methods generate corrections of different qualities. In this example, a correction using TOPUP from the software package FSL is of lower quality than both DL model 400 corrections (i.e., higher residual difference). The example also shows the performance for the DL models 400, which were trained respectively on fast ref scans and high-quality ref scans (i.e., “DL Fast Ref Scan” vs “DL HQ Ref Scan”). From the residual signals in the frontal cortex, it is apparent that the training with high quality reference data improves general BUDA reconstruction performance for conventionally acquired DWI data.

At Act A120, the training data is input into the network which outputs an estimated displacement field 430. In an embodiment, the DL model 400 is trained/configured to estimate a displacement field 430 when input reference data. Susceptibility artifacts in EPI generally manifest as distortions along the PE direction, that can be parametrized by a unidirectional displacement field shifting pixel coordinates. The displacement field U maps the distorted image I₁ to the corrected image I_{c}, while −U maps the reversed-encoded image I₂ to the same corrected image:

$${I}_{c} = ( {I}_{1} ∘ ( Id + U ) ) \cdot {J}_{d e t} ( Id + U ) ,$$

$${I}_{c} = ( {I}_{2} ∘ ( Id - U ) ) \cdot {J}_{d e t} ( Id - U ) ,$$

where Id represents the identity transformation, and ∘ denotes spatial unwarping. The unwarping process I₁ ∘ (Id+U) can be implemented as taking pixel value of the distorted image I₁(x, y+U(x,y)) to generate the corrected pixel at (x,y), with the second dimension being PE direction. J_{det}(·) is the Jacobian determinant to correct for intensity variations caused by local expansion and compression of the image signal. The DL model 400 is trained to estimate one or more of the following: a displacement field, a single corrected image, or a corrected image and the displacement field. The configuration of the DL model 400 may be adapted using different architectures and loss functions depending on the output.

In an embodiment, the DL model 400 is trained to learn a mapping from the input reversed-PE images (I₁, I₂) to the displacement field U. With the estimated U, corrected images I_{c1} and I_{c2} may be generated according to above equations from I₁ and I₂, respectively. Additionally, the displacement field 430 may be used to correct other EPI images, e.g., with different b-values or diffusion directions in the DWI context, acquired with the same (or scaled) distortion pattern. Once the displacement field is known for a specific EPI readout module, it can get applied to any other EPI readout module by scaling according to the ratio of the pixel bandwidth (which determines sensitivity) along the phase-encoding direction. In DWI, since low b-value DWI images typically have higher SNR and shorter acquisition times, the displacement field 430 is estimated using a pair of low b-value images. The displacement field 430 is applied to correct high b-value images for the same subject.

For training the network, in an embodiment, unsupervised learning may be used, with the loss function consisting of two components:

$$L = {L}_{MSE} ( {I}_{c1} , {I}_{c 2} ) + λ {L}_{s m o o t h} ( U , M ) .$$

L_{MSE}(I_{c1}, I_{c2}) measures the mean square error (MSE) between the two corrected images. Lₛₘₒₒₜₕ(U,M) is a spatially weighted smoothness loss on the estimated field U:

$${L}_{smooth} ( U , M ) = \frac{1}{{N}_{x} {N}_{y}} {\sum }_{x , y} M ( x , y ) ( {( \frac{{\partial }^{2} U ( x , y )}{\partial {x}^{2}} )}^{2} + {( \frac{{\partial }^{2} U ( x , y )}{\partial {y}^{2}} )}^{2} ) .$$

The weighting matrix M is derived from the input images, adaptively regularizing the smoothness penalty across the image. In background regions with low signal intensity, dominated by noise, it enforces a stronger smoothness constraint to prevent noise from being transferred into the estimated displacement field 430. It is calculated as follows:

$$D = Gaussion ( max ( {I}_{1} , {I}_{2} ) ) ,$$

$$M = ( 1 - \frac{D}{max ( D )} ) ( 1 - a ) + a ,$$

where Gaussion(·) denotes a Gaussian blurring operator, and a is a parameter that controls the relative smoothness penalty in high-intensity regions.

In another embodiment, training is performed using a mixed specialized loss function, combining supervised and unsupervised losses. When a reference method is available to provide displacement field and/or corrected images, additional supervised losses can be calculated between the estimated displacement field and the reference displacement field and/or between the generated corrected images and the reference corrected images.

In another embodiment, a global smoothness penalty without spatial weighting is used. For a global smoothness loss, a smaller λ improves the similarity between corrected blip-up and blip-down images but the estimated displacement field 430 may be noisy in the case of low-SNR inputs, which introduces substantial noise and artifacts when it is applied to correct other images (e.g., other diffusion values and diffusion directions in DWI). A larger λ reduces noise propagation but may lead to suboptimal correction in regions with fast B0 variations. The proposed spatially weighted smoothness loss suppresses the noise transfer in the low-SNR background region with a stronger smoothness penalty while maintaining the distortion correction performance in high-SNR regions using a smaller smoothness loss.

At act A130, the trained network is output and/or stored for later use during a procedure. In an example of an implementation of the DL training, an unsupervised learning-based method was developed for EPI, with the model 400 for displacement field estimation from a reversed-PE image pair. A spatially weighted smoothness loss was used to improve robustness to noise while maintaining correction quality. The method was evaluated in multiple organs with diverse sequences and protocols, showing generalizable performance.

In another implementation, a diverse DL training dataset covering RT-specific anatomies, including the pelvis, head/neck, and brain was used as a training data set. Data was collected from 50 healthy volunteers using 1.5 T and 3 T MRI systems following protocols optimized for each anatomy based on clinical guidelines. This approach resulted in approximately 2 hours of data acquisition per participant, generating a total of 35 k slice pairs for training. Ground truth for DL training may be computed using a TopUp-like method. The DL model 400 was trained to estimate the B0 map from the input DWI pair. DWI images were corrected for distortion by applying unwarping and intensity correction using the estimated field. A combination of unsupervised loss (i.e., similarity between the corrected image pair) and supervised loss (i.e., similarity between the DL results and the targets) was used to optimize the DL model 400.

In an embodiment, motion between scans may be addressed. Motion is assumed to be small because of the short acquisition time of EPI; however, motion correction may improve the results. Further, a rigid transformation may be incorporated to be jointly estimated along with the displacement field 430. The trained network is described with respect to DWI, but the correction method may also be applied to other EPI-based MRI, such as functional MRI. With different protocols, different training datasets and mechanisms may be used.

In an embodiment, the trained network may be used for high quality reference scans, high quality DL distortion correction methods, among other uses. The trained network may provide high quality reference scans, where the integrated acquisition of high quality B0 maps may be used as a reference scan e.g., for DL inference. Integrated high-quality BUDA reference scans with a short echo time (reference TE) and/or interleaved averaging may be played out prior to actual DWI data acquisition with the conventional echo time (Acquisition TE). As the underlying B0 field does not depend on the TE, the field map will remain consistent but exhibit higher SNR compared to conventional BUDA data. Since a shorter echo time allows shortening the repetition time TR as well, further reduction of the total scan time will be possible.

For high quality DL distortion correction models 400, the trained network may be used for generation of high-quality B0 maps. High SNR ground truth B0 maps may then assist in generating high quality distortion correction models 400, based on BUDA acquisitions. The network optimally matches the high quality B0 reference data. This may also result in improved distortion correction performance by combining typical reference scans and the high-quality DL distortion correction models 400. In addition, the acquisition of a dataset with two TE values may allow the additional computation of quantitative T2 map, which is completely matched to the DWI data.

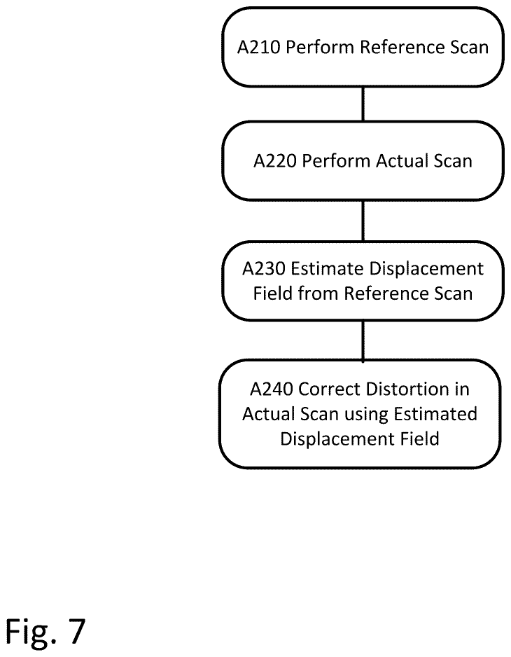

FIG. 7 depicts an example workflow for application of the trained network to new data acquired from a patient 15. The method is performed by the system of FIG. 1, 4, 9, or another system. The method is performed in the order shown or other orders. Additional, different, or fewer acts may be provided. In an embodiment, a patient scan is performed using a MR protocol in order to acquire MR data which is corrected using an estimated displacement field 430. As depicted and described in FIG. 1 above, the MR data may be acquired using a magnetic resonance apparatus 10. For example, gradient coils, a whole-body coil, and/or local coils generate a pulse or scan sequence in a magnetic field created by a main magnet or coil. The whole-body coil or local coils receive MR signals. Different objects, organs, or regions of a patient 15 may be scanned. The MR data is k-space data. The scan may provide 2D, 3D, or 4D (e.g., 3D plus time or different diffusion encodings, e.g., diffusion weightings (b-values) and/or diffusion directions) data for further analysis. The trained specialized DL model 400 may be trained/configured at any point prior to its application. The DL model 400 may be updated when new training datasets become available.

At act A210, a reference scan is performed on a patient 15. In a conventional Blip-Up Blip-Down Acquisition (BUDA), the echo time (TE) is typically matched to the TE used in the actual EPI data acquisition. The matching between the BUDA pair images is highly contrast dependent. The separate blip-up acquisition needs to employ the same TE, as the remaining data acquisition, thereby limiting available SNR. The reference scan is therefore adapted to include a high-quality BUDA reference scan in order to acquire a blip-reversed reference dataset for example as described in FIG. 3. The high-quality BUDA reference scan is performed with a low echo time (Reference TE) and/or interleaved averaging that is performed for example directly prior to or at the beginning of the actual DWI data acquisition with the conventional echo time (Acquisition TE). The Acquisition TE for DWI may be, for example, between 60-100 ms. For different types of scans, e.g., fMRI, diffusion, or structural EPI, and the anatomical region being imaged, different Acquisition TE values may be used. In an embodiment, the reference TE is shorter than the Acquisition TE, for example, 40 ms or less, less than 30 ms, less than 25 ms, or less than 20 ms. Alternatively, the TE of the reference scan may be longer, for example in the context of Blood Oxygenation Level Dependent (BOLD) imaging. BOLD is performed using T2*-weighted EPI (i.e., gradient-echo like EPI with no refocusing pulse), and intra-voxel dephasing which may lead to signal loss in locations with strong field deviations. In this case, it might turn out beneficial to acquire the reference scans with T2-weighted EPI (i.e., spin-echo EPI with a refocusing pulse). While the TE is longer (due to the additional refocusing), these images will not suffer from local signal loss and thus allow for a better precision of the displacement map estimation in those regions. The scan is thus optimized so that the data can also be used to infer the displacement field 430 outside of the brain. The reference TE is thus set in such a way that it is applicable for alternative body regions, for example, full body scans. As the underlying B0 field does not depend on the TE, the field map remains consistent but exhibits higher SNR compared to conventional BUDA data. Since a shorter echo time allows shortening the repetition time TR as well, further reduction of the total scan time may be possible. In an embodiment, multiple iterations of the reference acquisition may be performed and averaged. This approach may increase the reference acquisition time but will generate a blip-reversed reference dataset of even higher quality.

At act A220, an actual imaging scan is performed on the patient 15. The imaging scan may be performed subsequent to the reference scan using an Acquisition TE that is different than the reference TE. The imaging scan is performed using an MR apparatus 10 such as described in FIG. 1. Different MR protocols may be used for the scan of a patient 15. Examples described herein use DWI, but the techniques for distortion correction may be applied to alternative protocols or techniques. These protocols may be applied to both the reference scan and imaging scan where applicable. MR protocols include a variety of pulse sequences tailored to specific diagnostic purposes. Common examples include T1-weighted imaging (for anatomical detail and tissue contrast), T2-weighted imaging (highlighting fluid or edema), fluid-attenuated inversion recovery (FLAIR, sensitive to pathology), diffusion-weighted imaging (DWI) for assessing water mobility in tissues, diffusion tensor imaging (DTI, assessing white matter integrity), functional MRI (fMRI, detecting brain activation patterns), and susceptibility-weighted imaging (SWI, visualizing blood and iron content). Additional protocols include MR angiography (MRA, imaging blood vessels), spectroscopy (MRS, analyzing tissue metabolites), and gradient-echo sequences (GRE, rapid imaging, sensitive to susceptibility effects).

In an embodiment, the MR protocol is for DWI. In DWI, specialized gradient waveforms are applied to impart sensitivity to the microscopic diffusion of water molecules within biological tissues. These gradient waveforms play a central role in encoding diffusion information into the MRI signal, allowing differentiation of tissue structures based on their unique water diffusion characteristics. The gradient waveform used in diffusion MRI sequences may include two strong gradient pulses of equal amplitude, shape, and duration, separated by a controlled time interval. One widely adopted gradient waveform is the Stejskal-Tanner waveform, which consists of two gradient pulses, for example of opposite polarity along a selected spatial axis, separated by a diffusion time interval (often called the diffusion encoding time). When these paired gradient pulses are applied, water molecules undergoing random Brownian motion experience a phase dispersion proportional to the gradient amplitude, duration, interval between gradient pulses, and their diffusion characteristics. Consequently, stationary water molecules accumulate no net phase shift between gradient pulses, while diffusing water molecules accumulate significant phase dispersion, resulting in attenuation of measured MRI signals. The result of the patient scan is MR data acquired using a specific MR protocol. The MR data, however, is uncorrected and may be degraded by distortions in the B0 field during acquisition.

At act A230, the trained specialized DL model 400 is applied to the reference scan to determine an estimated displacement field 430. The trained model 400 may include or be the model 400 described in FIG. 4. The network of the trained specialized DL model 400 provides an estimate of the displacement field 430 that is used for distortion correction of the image data acquired by the actual imaging scan of the patient 15. In an example of using EPI, the distortions vary spatially, with large displacements in regions of strong susceptibility effects. Existing DL-based correction methods predominantly rely on U-Net architectures that use fixed 3×3 convolutional kernels with local receptive fields. While effective for many tasks, this design limits the network's ability to handle large, non-uniform displacements. To address this, the trained specialized DL model 400 incorporates deformable convolutional modulation blocks, allowing the receptive field to dynamically adjust and the features to be modulated based on local inputs. The trained specialized DL model 400 outperforms typical U-Net-based models, particularly in high-resolution images with severe distortions.

The correction method uses two EPI images with reversed phase encoding (PE) directions. In DWI, acquiring such pairs for every b-value and diffusion direction is time-consuming, especially for high b-value DWI with low SNR requiring multiple averages or for large numbers of diffusion directions. To improve efficiency and to ensure a consistent unwarping independent of diffusion weighting or direction, the displacement field 430 from at least one pair of reversed-PE low b-value DWI is acquired and applied to other single-PE images. This necessitates a generalizable displacement field 430. While lower smoothness loss achieves better similarity metrics in correcting the input low b-value images, the estimated displacement fields transfer noise from the inputs to the high b-value images. Conversely, a stronger smoothness penalty prevents the displacement field 430 from being noisy, but compromises correction performance. To improve this trade-off, a spatially varying smoothness loss weighted by input intensities is used that achieves a better balance by enforcing stronger smoothness penalty in low-SNR background regions while applying a smaller smoothness loss in high-SNR regions.

In an embodiment, the image data acquired is 2D. The trained model 400 is likewise trained and configured to generate a 2D displacement field 430. In another embodiment, the trained model 400 may be configured to use adjacent (neighboring) 2D slices in its estimation or to directly use a 3D volume. This process may result in 3D spatial awareness. The output may be a 2D displacement field 430 or a 3D displacement field 430.

At act A240, the estimated displacement field 430 is applied to correct the image scan data. The trained specialized DL model 400 may be configured to both estimate the displacement field 430 and correct the image scan data. In an embodiment, image unwarping and Jacobian modulation is applied using the estimated displacement field 430. The distortions in the image scan data are corrected using an estimated displacement field 430 that maps distorted image coordinates back to their true anatomical locations. Image unwarping involves using the displacement field 430 to resample the distorted image data into an undistorted space. For each voxel/pixel in the target (undistorted) space, the displacement field 430 is used to locate the corresponding point in the distorted image. Interpolation is then applied to retrieve the intensity value from the distorted image at that location. This process reconstructs an anatomically accurate image representation. The Jacobian modulation provides that the intensity values remain physically meaningful after resampling. The Jacobian determinant of the deformation field quantifies local changes introduced by the unwarping transformation. Without modulation, regions compressed or expanded by the distortion could appear artificially brighter or dimmer. By scaling the resampled intensities with the Jacobian determinant, signal intensity is adjusted to compensate for these geometric changes, preserving quantitative accuracy in diffusion metrics. Together, image unwarping and Jacobian modulation correct both spatial and intensity distortions in the image scan data. In an embodiment, the specialized DL model may be configured and trained to output the corrected image directly.

In an embodiment, if the images are noisy and there is information about the noise, there may be an additional step which applies a denoiser algorithm on the input images. In another embodiment, the displacement field 430 may be filtered prior to correcting the image scan data. For example, a displacement field refinement module may be used to refine the displacement field 430 prior to correction of the scan data. In another embodiment, motion between the input pairs may be detected/corrected for.

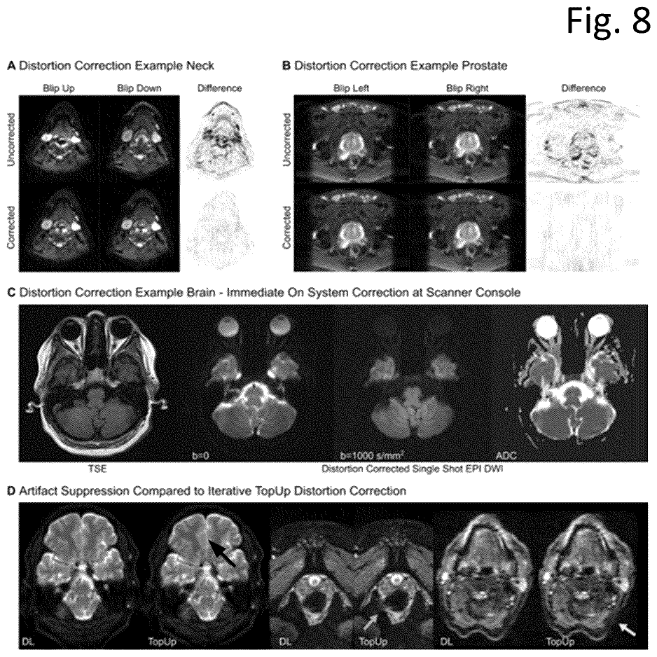

The resulting trained specialized DL model 400 provides stable distortion correction performance across various anatomies. FIG. 8 depicts several example results from the DL BUDA Distortion Correction using a combined approach. In (A), a distortion correction example in the neck is depicted where the B0 estimation uses two phase-encoding directions (e.g., blip-up/down). After correction, the input images align well, with residual differences primarily representing noise. In (B) a distortion correction example in the prostate is depicted. In this example, after correction, the input images align well, with residual differences primarily representing noise. In (C) the proposed DL distortion correction method is integrated into a DWI sequence, enabling immediate correction-directly at the scanner. The method effectively corrected distortions in regions prone to severe artifacts, such as the temporal lobe poles and eyes. A distortion-free TSE image is shown for anatomical reference. (D) depicts a comparison between the specialized DL model 400 and a conventional algorithm. The DL method may outperform the widely used conventional algorithm for B0 distortion correction. In the brain, the conventional algorithm introduced ringing artifacts between hemispheres (arrow). In the pelvis, the conventional algorithm caused significant blurring of the rectal wall (arrow). In the neck, the conventional algorithm failed to reconstruct the skin surface (arrow). In contrast, the DL method avoided these artifacts, delivering stable and consistent correction across all cases.

In addition, the trained specialized DL model 400 achieves significantly faster inference than conventional algorithms, processing approximately (0.120 s/vol), compared to iterative methods, such as the above described conventional algorithm (24.2 min/vol). This >10,000× speed-up enables real-time distortion correction directly at the scanner, using a sequence with one preceding reversed PE EPI volume and the DL method. Further, the here proposed method achieved better distortion corrected than the conventional algorithm in various cases.

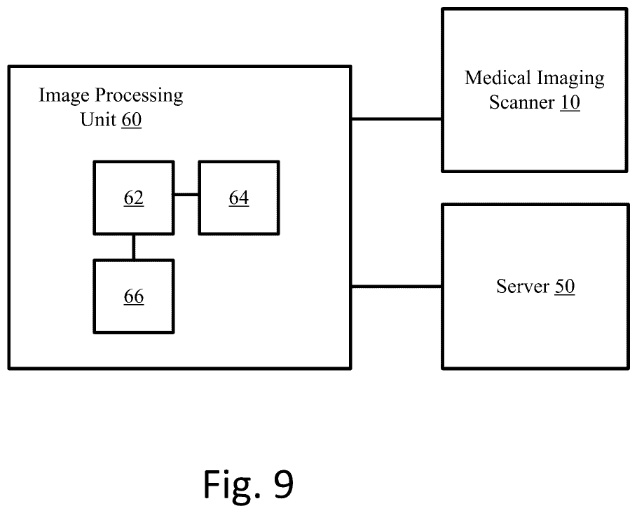

FIG. 9 depicts an example image processing unit 60 for implementing the trained specialized DL model 400. The system is configured to perform the method of FIGS. 5, 7, and other acts described herein. In FIG. 9, the image processing unit 60 includes a processor 62, memory 64, and interface 66. The image processing unit 60 is in communication with a medical imaging device 10, for example as described in FIG. 1, and a server 50. The medical imaging device is configured to acquire MR imaging data, for example k-space data that is reconstructed into an MR image of an organ or a region of a patient 15. The image processing unit 60 is configured to acquire reference data which is used to correct distortions in the MR image. The image processing unit 60 may further be configured to reconstruct the image from the MR data acquired by the medical imaging device 10. The reconstructed image may be a two-dimensional distribution of pixels representing an area of the patient 15 and/or a three-dimensional distribution of voxels representing a volume of the patient 15. The memory 64 is configured to store instructions and parameters for the model(s) 400. The interface 66 is configured to display the images, etc. and/or accept inputs from a user. The server 50 may perform similar tasks as the image processing unit 60 and/or may provide additional processing, storage, or analysis for example using a cloud-based platform.

The processor 62 may include an image processor that generates a reconstructed image, for example using a machine learning network (machine learning model 400). The image processor is a general processor, digital signal processor, three-dimensional data processor, graphics processing unit, application specific integrated circuit, field programmable gate array, artificial intelligence processor, digital circuit, analog circuit, combinations thereof, or another now known or later developed device for displacement field 430 estimation, distortion correction, and/or image generation. The image processor is a single device, a plurality of devices, or a network. For more than one device, parallel or sequential division of processing may be used. Different devices making up the image processor may perform different functions. In one embodiment, the image processor is also a control processor or other processor of the imaging device. Other image processors of the imaging device or external to the imaging device may be used. The image processor is configured by software, firmware, and/or hardware to process the data acquired by the imaging device and output one or more images.

The interface 66 includes an input device and an output device. The input may be an interface, such as interfacing with a computer network, memory, database, medical image storage, or other source of input data. The input may be a user input device, such as a mouse, trackpad, keyboard, roller ball, touch pad, touch screen, or another apparatus for receiving user input. The output is a display device but may be an interface. The display is a CRT, LCD, plasma, projector, printer, or other display device. The display is configured by loading an image to a display plane or buffer. The display is configured to display a reconstructed image(s) of the region of the patient 15. The interface 66 may include a graphical user interface (GUI) enabling user interaction with the medical imaging device and enables user modification or selections in substantially real time.

The instructions for implementing the processes, methods, and/or techniques discussed herein are provided on non-transitory computer-readable storage media or memories, such as a cache, buffer, RAM, removable media, hard drive, or other computer readable storage media, for example the memory 64. The instructions are executable by the processor or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

In an embodiment, the processor 62 implements one or more machine learning networks that are stored in the memory in order to provide displacement field 430 estimation and/or distortion correction. In particular, a machine learning network may comprise a neural network, a support vector machine, a decision tree and/or a Bayesian network, and/or the machine learning network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network, a generative network, and/or a generative adversarial network. In an embodiment, the image reconstruction framework is provided by or implemented with a neural network trained using deep learning. The network(s) may be defined as a plurality of sequential feature units or layers. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction of the input data. The features are learned to reconstruct lower-level features (i.e., features at a more abstract or compressed level). For example, features for generating a fused image or higher resolution image are learned. For a next unit, features for reconstructing the features of the previous unit are learned, providing more abstraction. Each node of the unit represents a feature. Different units are provided for learning different features.

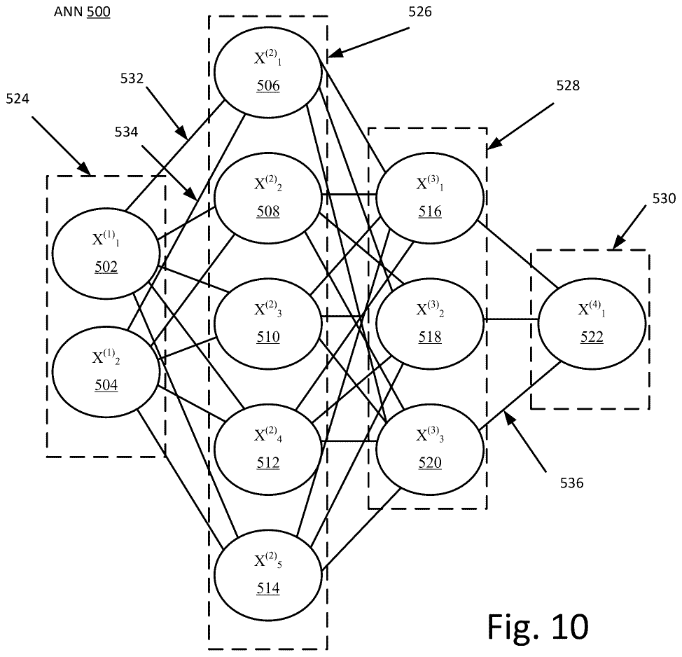

Various units or layers may be used, such as convolutional, pooling (e.g., max-pooling), deconvolutional, fully connected, or other types of layers. Within a unit or layer, any number of nodes is provided. For example, 100 nodes are provided. Later or subsequent units may have more, fewer, or the same number of nodes. In general, for convolution, subsequent units have more abstraction. FIG. 10 shows an embodiment of an artificial neural network (ANN) 500, in accordance with one or more embodiments. Alternative terms for “artificial neural network” are “neural network”, “artificial neural net” or “neural net”. The artificial neural network 500 may be used in part in, for example, the one or more machine learning based networks utilized for the displacement field 430 estimation and/or distortion correction, etc.

The artificial neural network 500 includes nodes 502-522 and edges 532, 534, . . . , 536, wherein each edge 532, 534, . . . , 536 is a directed connection from a first node 502-522 to a second node 502-522. In general, the first node 502-522 and the second node 502-522 are different nodes 502-522, it is also possible that the first node 502-522 and the second node 502-522 are identical. For example, in FIG. 10, the edge 532 is a directed connection from the node 502 to the node 506, and the edge 534 is a directed connection from the node 504 to the node 506. An edge 532, 534, . . . , 536 from a first node 502-522 to a second node 502-522 is also denoted as “ingoing edge” for the second node 502-522 and as “outgoing edge” for the first node 502-522.

In this embodiment, the nodes 502-522 of the artificial neural network 500 may be arranged in layers 524-530, wherein the layers may include an intrinsic order introduced by the edges 532, 534, . . . , 536 between the nodes 502-522. In particular, edges 532, 534, . . . , 536 may exist only between neighboring layers of nodes. In the embodiment shown in FIG. 10, there is an input layer 524 including only nodes 502 and 504 without an incoming edge, an output layer 530 including only node 522 without outgoing edges, and hidden layers 526, 528 in-between the input layer 524 and the output layer 530. In general, the number of hidden layers 526, 528 may be chosen arbitrarily. The number of nodes 502 and 504 within the input layer 524 usually relates to the number of input values of the neural network 500, and the number of nodes 522 within the output layer 530 usually relates to the number of output values of the neural network 500.

In particular, a (real/complex) number may be assigned as a value to every node 502-522 of the neural network 500. Here, x⁽ⁿ⁾ᵢ denotes the value of the i-th node 502-522 of the n-th layer 524-530. The values of the nodes 502-522 of the input layer 524 are equivalent to the input values of the neural network 500, the value of the node 522 of the output layer 530 is equivalent to the output value of the neural network 500. Furthermore, each edge 532, 534, . . . , 536 may include a weight being a real number, in particular, the weight is a real number within the interval [−1, 1] or within the interval [0, 1]. Here, w^{(m,n)}_{i,j} denotes the weight of the edge between the i-th node 502-522 of the m-th layer 524-530 and the j-th node 502-522 of the n-th layer 524-530. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 502-522 of the (n+1)-th layer 524-530 may be calculated based on the values of the nodes 502-522 of the n-th layer 524-530 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 524 are given by the input of the neural network 500, wherein values of the first hidden layer 526 may be calculated based on the values of the input layer 524 of the neural network, wherein values of the second hidden layer 528 may be calculated based in the values of the first hidden layer 526, etc.

In order to set the values w^{(m,n)}_{i,j} for the edges, the neural network 500 has to be trained using training data. In particular, training data includes training input data and training output data (denoted as tᵢ). For a training step, the neural network 500 is applied to the training input data to generate calculated output data. In particular, the training data and the calculated output data include a number of values, said number being equal with the number of nodes of the output layer.

In particular, a comparison between the calculated output data and the training output data is used to recursively adapt the weights within the neural network 500 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾j may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾j, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 530, wherein f′ is the first derivative of the activation function, and t⁽ⁿ⁺¹⁾j is the comparison training value for the j-th node of the output layer 530.

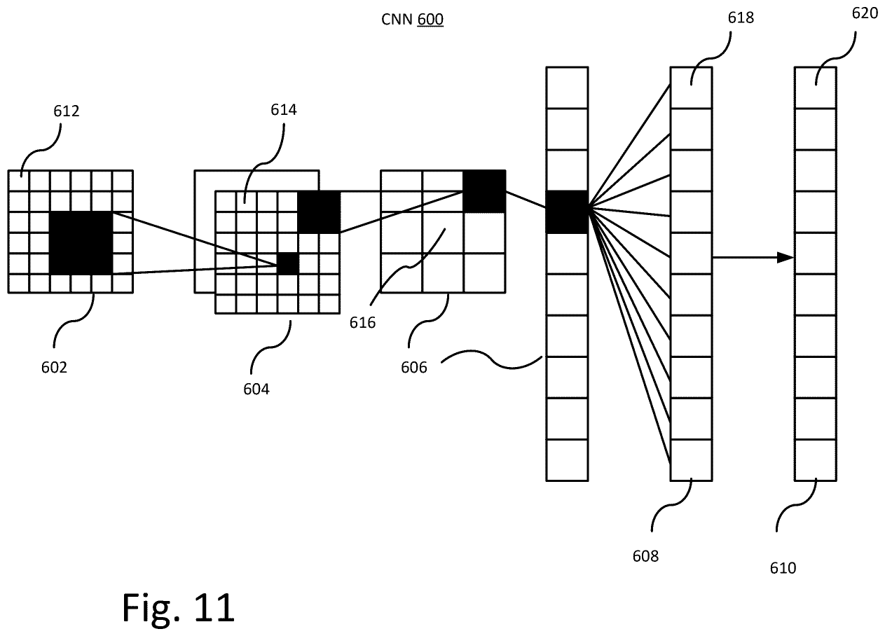

FIG. 11 shows a convolutional neural network (CNN) 600, in accordance with one or more embodiments. Machine learning networks described herein, such as, e.g., for the displacement field 430 estimation and/or distortion correction etc. may be implemented using techniques or components described by the convolutional neural network 600.

In the embodiment shown in FIG. 11 the convolutional neural network 600 includes an input layer 602, a convolutional layer 604, a pooling layer 606, a fully connected layer 608, and an output layer 610. Alternatively, the convolutional neural network 600 may include several convolutional layers 604, several pooling layers 606, and several fully connected layers 608, as well as other types of layers. The order of the layers may be chosen arbitrarily, usually fully connected layers 608 are used as the last layers before the output layer 610.

In particular, within a convolutional neural network 600, the nodes 612-620 of one layer 602-610 may be considered to be arranged as a d-dimensional matrix or as a d-dimensional image. In particular, in the two-dimensional case the value of the node 612-620 indexed with i and j in the n-th layer 602-610 may be denoted as x⁽ⁿ⁾_{[i,j]}. However, the arrangement of the nodes 612-620 of one layer 602-610 does not have an effect on the calculations executed within the convolutional neural network 600 as such, since these are given solely by the structure and the weights of the edges.

In particular, a convolutional layer 604 is characterized by the structure and the weights of the incoming edges forming a convolution operation based on a certain number of kernels. In particular, the structure and the weights of the incoming edges are chosen such that the values x⁽ⁿ⁾k of the nodes 614 of the convolutional layer 604 are calculated as a convolution x⁽ⁿ⁾k=Kₖ*x⁽ⁿ⁻¹⁾ based on the values x⁽ⁿ⁻¹⁾ of the nodes 612 of the preceding layer 602, where the convolution * is defined in the two-dimensional case as:

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 612-618 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 612-620 in the respective layer 602-610. In particular, for a convolutional layer 604, the number of nodes 614 in the convolutional layer may be equivalent to the number of nodes 612 in the preceding layer 602 multiplied with the number of kernels.

If the nodes 612 of the preceding layer 602 are arranged as a d-dimensional matrix, using a plurality of kernels may be interpreted as adding a further dimension (denoted as “depth” dimension), so that the nodes 614 of the convolutional layer 604 are arranged as a (d+1)-dimensional matrix. If the nodes 612 of the preceding layer 602 are already arranged as a (d+1)-dimensional matrix including a depth dimension, using a plurality of kernels may be interpreted as expanding along the depth dimension, so that the nodes 614 of the convolutional layer 604 are arranged also as a (d+1)-dimensional matrix, wherein the size of the (d+1)-dimensional matrix with respect to the depth dimension may be a factor of the number of kernels larger than in the preceding layer 602.

The advantage of using convolutional layers 604 is that spatially local correlation of the input data may exploited by enforcing a local connectivity pattern between nodes of adjacent layers, in particular by each node being connected to only a small region of the nodes of the preceding layer.

In the embodiment shown in FIG. 11, the input layer 602 includes 36 nodes 612, arranged as a two-dimensional 6×6 matrix. The convolutional layer 604 includes 72 nodes 614, arranged as two two-dimensional 6×6 matrices, each of the two matrices being the result of a convolution of the values of the input layer with a kernel. Equivalently, the nodes 614 of the convolutional layer 604 may be interpreted as arranged as a three-dimensional 6×6×2 matrix, wherein the last dimension is the depth dimension.

A pooling layer 606 may be characterized by the structure and the weights of the incoming edges and the activation function of its nodes 616 forming a pooling operation based on a non-linear pooling function f. For example, in the two-dimensional case the values x⁽ⁿ⁾ of the nodes 616 of the pooling layer 606 may be calculated based on the values x⁽ⁿ⁻¹⁾ of the nodes 614 of the preceding layer 604 as

$${x}^{( n )} [ i , j ] = f ( {x}^{( n - 1 )} [ {id}_{1} , {jd}_{2} ] , … , {x}^{( n - 1 )} [ {id}_{1} + {d}_{1} - 1 , {jd}_{2} + {d}_{2} - 1 ] )$$

In other words, by using a pooling layer 606, the number of nodes 614, 616 may be reduced, by replacing a number d1·d2 of neighboring nodes 614 in the preceding layer 604 with a single node 616 being calculated as a function of the values of said number of neighboring nodes in the pooling layer. In particular, the pooling function f may be the max-function, the average, or the L2-Norm. In particular, for a pooling layer 606 the weights of the incoming edges are fixed and are not modified by training.

The advantage of using a pooling layer 606 is that the number of nodes 614, 616 and the number of parameters is reduced. This leads to the amount of computation in the network being reduced and to a control of overfitting.

In the embodiment shown in FIG. 11, the pooling layer 606 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 9.

A fully-connected layer 608 may be characterized by the fact that a majority, in particular, all edges between nodes 616 of the previous layer 606 and the nodes 618 of the fully-connected layer 608 are present, and wherein the weight of each of the edges may be adjusted individually.

In this embodiment, the nodes 616 of the preceding layer 606 of the fully-connected layer 608 are displayed both as two-dimensional matrices, and additionally as non-related nodes (indicated as a line of nodes, wherein the number of nodes was reduced for a better presentability). In this embodiment, the number of nodes 618 in the fully connected layer 608 is equal to the number of nodes 616 in the preceding layer 606. Alternatively, the number of nodes 616, 618 may differ.

A convolutional neural network 600 may also include a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks may be wired using summation (residual/dense neural networks), element-wise multiplication (attention) or other differentiable operators. Therefore, the convolutional neural network architecture may be nested rather than being sequential if the whole pipeline is differentiable.

In particular, convolutional neural networks 600 may be trained based on the backpropagation algorithm. For preventing overfitting, methods of regularization may be used, e.g. dropout of nodes 612-620, stochastic pooling, use of artificial data, weight decay based on the L1 or the L2 norm, or max norm constraints. Different loss functions may be combined for training the same neural network to reflect the joint training objectives. A subset of the neural network parameters may be excluded from optimization to retain the weights pretrained on another datasets.

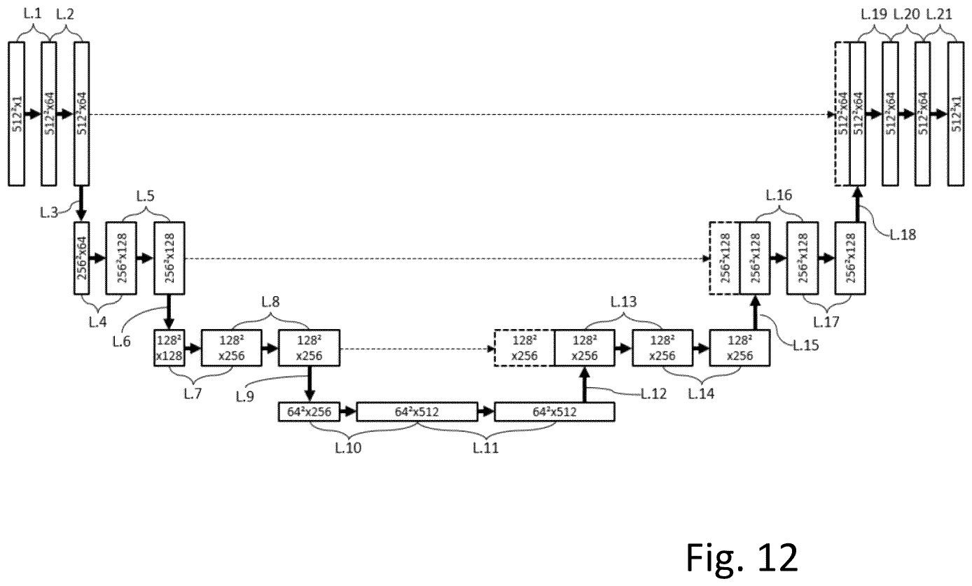

In an embodiment, the diffusion model is based on is a convolutional neural network, in particular, a convolutional neural network having a U-net structure, for example as displayed in FIG. 12. In FIG. 12, the input data to the machine learning network is a two-dimensional medical image comprising 512×512 pixel, every pixel comprising one intensity value. The machine learning network comprises convolutional layers (indicated by solid, horizontal arrows), pooling layers (indicating by solid arrows pointing down), and upsampling layers (indicated by solid arrows pointing up), the number of the respective nodes is indicated within the boxes. Within the U-net structure first the input images are downsampled (decreasing the size of the images and increasing the number of channels), afterwards they are upsampled (increasing the size of the images and decreasing the number of channels) to generate a transformed image.

All except the last convolutional layers L.1, L.2, L.4, L.5, L.7, L.8, L.10, L.11, L.13, L.14, L.16, L.17, L.19, L.20 use 3×3 kernels with a padding of 1, the ReLU activation function, and a number of filters/convolutional kernels that matches the number of channels of the respective node layers as indicated in FIG. 12. The last convolutional layer L.21 uses a 1×1 kernel with no padding and the ReLU activation function.

The pooling layers L.3, L.6, L.9 are max-pooling layers, replacing four neighboring nodes with only one node, the value being the maximum of the values of the four neighboring nodes. The upsampling layers L. 12, L.15, L.18 are transposed convolution layers with 3×3 kernels and stride 2, which effectively quadruple the number of nodes. The dashed horizontal arrows correspond to concatenation operations, where the output of a convolutional layer L.2, L.5, L.8 of the downsampling branch of the U-net structure is used as additional inputs for a convolutional layer L.13, L.16, L.19 of the upsampling branch of the U-net structure. This additional input data is treated as additional channels in the input node layer for the convolutional layer L.13, L.16, L.19 of the upsampling branch.

The networks described herein may use one or more of a ConvFormer block and/or a Def-ConvFormer block. A ConvFormer block is a hybrid architecture designed to merge the strengths of convolutional neural networks (CNNs) and transformers for vision tasks. The ConvFormer block begins with a depth wise convolution, which efficiently captures local spatial patterns while maintaining computational efficiency. This convolution introduces an inductive bias that helps the model 400 generalize better, especially with limited data. Following the convolution, a normalization layer, often layer normalization, is applied to stabilize the learning process and improve convergence. Next, the ConvFormer block includes a lightweight self-attention mechanism. The output then passes through a feedforward network, which may use pointwise convolutions and non-linear activations such as GELU to enhance representation learning. Residual connections span across the main layers of the block, ensuring gradient flow and preserving information from earlier stages.

The Def-ConvFormer block is an enhanced variant of the ConvFormer architecture that introduces deformable convolutions to improve spatial adaptability in visual processing. Unlike standard convolutions that operate on fixed grid patterns, deformable convolutions allow the network to dynamically adjust its sampling locations based on the input content. This flexibility enables the model 400 to better capture complex geometric transformations and object deformations in images. In a Def-ConvFormer block, the input first passes through a deformable convolution layer, which enhances local feature extraction by learning spatial offsets that adapt to the image structure. This may be followed by or preceded by normalization, for example via layer normalization, to stabilize training. The block then mimics self-attention mechanism with a Hadamard product, allowing the network to model longer-range dependencies while maintaining computational efficiency. A feedforward network follows, often implemented with pointwise convolutions and non-linear activations, to refine the features. Throughout the block, residual connections preserve input information and ensure smooth gradient flow. By integrating deformable convolution into the hybrid ConvFormer design, the Def-ConvFormer block improves the network's ability to handle spatially varying distortions.

While the invention has been described above by reference to various embodiments, many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

The following is a list of non-limiting illustrative embodiments disclosed herein: Illustrative embodiment 1: system for deep learning based distortion correction, the system comprising: a medical imaging scanner configured to acquire a blip-reversed reference dataset and an acquisition dataset of a region of a patient; and an image processing unit configured to estimate a displacement field based on the blip-reversed reference dataset using a deep learning model, wherein the deep learning model comprises an encoder-decoder architecture with one or more deformable convolutional modulation blocks; the image processing unit configured to correct the acquisition dataset using the estimated displacement field.

Illustrative embodiment 2. The system of a previous illustrative embodiment, wherein the blip-reversed reference dataset comprises a high-quality reference scan acquired using a reference TE that is different than an Acquisition TE used to acquire the acquisition dataset.

Illustrative embodiment 3. The system of a previous illustrative embodiment, wherein the blip-reversed reference dataset is acquired as two or more separate scans comprising blip-up and blip-down using the reference TE.

Illustrative embodiment 4. The system of a previous illustrative embodiment, wherein adapted data averaging is performed in an interleaved fashion between phase encoding directions.

Illustrative embodiment 5. The system of a previous illustrative embodiment, wherein the region comprises a region other than a brain of the patient.

Illustrative embodiment 6. The system of a previous illustrative embodiment, wherein the deep learning model comprises a plurality of resolution layers comprising the one or more deformable convolutional modulation blocks and a down sampling block or up sampling block, wherein a residual connection to an input is made in a last resolution level, wherein a 1×1 convolution is used to estimate the displacement field, wherein a spatial transformer block is used for unwarping and Jacobian modulation for intensity correction of the acquisition dataset using the estimated displacement field.

Illustrative embodiment 7. The system of a previous illustrative embodiment, wherein the deep learning model is trained at least partially on blip-reversed reference data.

Illustrative embodiment 8. The system of a previous illustrative embodiment, further comprising: displaying, by a display, an image of the corrected acquisition dataset.

Illustrative embodiment 9. The system of a previous illustrative embodiment, wherein the image processing unit is part of the medical imaging scanner, wherein the distortion correction is performed at a scanner console.

Illustrative embodiment 10. A method for training a network for deep learning based distortion correction, the method comprising: acquiring a training dataset comprising pairs of blip-up and down reference data; iteratively training the network to estimate a displacement field for use in distortion correction of acquired image data for a patient when input a respective pair of blip-up and blip down reference data, the network comprising an encoder-decoder architecture with one or more deformable convolutional modulation blocks, the training comprising minimizing a loss function comprising a similarity loss between two corrected images and a spatially weighted smoothness loss on the estimated displacement field; and storing the trained network.

Illustrative embodiment 11. The method of a previous illustrative embodiment, wherein the training dataset comprises a multi-organ dataset, including brain and other body parts.

Illustrative embodiment 12. The method of a previous illustrative embodiment, wherein the network comprises a plurality of resolution layers comprising a Def-ConvFormer block and a down sampling block or up sampling block, wherein a residual connection to the input is made in a last resolution level, wherein a 1×1 convolution is used to estimate the displacement field, wherein a spatial transformer block is used for unwarping and Jacobian modulation for intensity correction using the estimated displacement field.

Illustrative embodiment 13. The method of a previous illustrative embodiment, wherein the pairs of reference data are generated by a high quality reference scan acquired using a reference TE that is different from Acquisition TE used to acquire the acquired image data.

Illustrative embodiment 14. The method of a previous illustrative embodiment, wherein the loss function further comprises a supervised loss on the estimated displacement field and the corrected images.

Illustrative embodiment 15. A method for deep learning enabled B0 mapping, the method comprising: acquiring, by a medical imaging scanner, a blip-reversed reference dataset; acquiring, by the medical imaging scanner, an acquisition dataset of a region of a patient; estimating, using a deep learning model, a displacement field based on the blip-reversed reference dataset, the deep learning model comprising encoder-decoder architecture with one or more deformable convolutional modulation blocks; and correcting the acquisition dataset using the displacement field.

Illustrative embodiment 16. The method of a previous illustrative embodiment, wherein acquiring the blip-reversed reference dataset uses a reference TE that is different from an Acquisition TE used to acquire the acquisition dataset of the patient.

Illustrative embodiment 17. The method of a previous illustrative embodiment, wherein the medical imaging scanner comprises a magnetic resonance scanner configured to perform EPI DWI.

Illustrative embodiment 18. The method of a previous illustrative embodiment, wherein the region comprises a region other than a brain of the patient.

Illustrative embodiment 19. The method of a previous illustrative embodiment, wherein the deep learning model comprises a plurality of resolution layers comprising a Def-ConvFormer block and a down sampling block or up sampling block, wherein a residual connection to an input is made in a last resolution level, wherein a 1×1 convolution is used to estimate the displacement field, wherein a spatial transformer block is used for unwarping and Jacobian modulation for intensity correction of the acquisition dataset using the estimated displacement field.

Illustrative embodiment 20. The method of a previous illustrative embodiment, wherein the deep learning model is trained at least partially on blip-reversed reference scan data.

1. A system for deep learning based distortion correction, the system comprising:
a medical imaging scanner configured to acquire a blip-reversed reference dataset and an acquisition dataset of a region of a patient; and
an image processing unit configured to estimate a displacement field based on the blip-reversed reference dataset using a deep learning model, wherein the deep learning model comprises an encoder-decoder architecture with one or more deformable convolutional modulation blocks;
the image processing unit configured to correct the acquisition dataset using the estimated displacement field.

2. The system of claim 1, wherein the blip-reversed reference dataset comprises a high-quality reference scan acquired using a reference TE that is different than an Acquisition TE used to acquire the acquisition dataset.

3. The system of claim 2, wherein the blip-reversed reference dataset is acquired as two or more separate scans comprising blip-up and blip-down using the reference TE.

4. The system of claim 2, wherein adapted data averaging is performed in an interleaved fashion between phase encoding directions.

5. The system of claim 1, wherein the region comprises a region other than a brain of the patient.

6. The system of claim 1, wherein the deep learning model comprises a plurality of resolution layers comprising the one or more deformable convolutional modulation blocks and a down sampling block or up sampling block, wherein a residual connection to an input is made in a last resolution level, wherein a 1×1 convolution is used to estimate the displacement field, wherein a spatial transformer block is used for unwarping and Jacobian modulation for intensity correction of the acquisition dataset using the estimated displacement field.

7. The system of claim 1, wherein the deep learning model is trained at least partially on blip-reversed reference data.

8. The system of claim 1, further comprising:
displaying, by a display, an image of the corrected acquisition dataset.

9. The system of claim 1, wherein the image processing unit is part of the medical imaging scanner, wherein the distortion correction is performed at a scanner console.

10. A method for training a network for deep learning based distortion correction, the method comprising:
acquiring a training dataset comprising pairs of blip-up and down reference data;
iteratively training the network to estimate a displacement field for use in distortion correction of acquired image data for a patient when input a respective pair of blip-up and blip down reference data, the network comprising an encoder-decoder architecture with one or more deformable convolutional modulation blocks, the training comprising minimizing a loss function comprising a similarity loss between two corrected images and a spatially weighted smoothness loss on the estimated displacement field; and
storing the trained network.

11. The method of claim 10, wherein the training dataset comprises a multi-organ dataset, including brain and other body parts.

12. The method of claim 10, wherein the network comprises a plurality of resolution layers comprising a Def-ConvFormer block and a down sampling block or up sampling block, wherein a residual connection to the input is made in a last resolution level, wherein a 1×1 convolution is used to estimate the displacement field, wherein a spatial transformer block is used for unwarping and Jacobian modulation for intensity correction using the estimated displacement field.

13. The method of claim 10, wherein the pairs of reference data are generated by a high quality reference scan acquired using a reference TE that is different from Acquisition TE used to acquire the acquired image data.

14. The method of claim 10, wherein the loss function further comprises a supervised loss on the estimated displacement field and the corrected images.

15. A method for deep learning enabled B0 mapping, the method comprising:
acquiring, by a medical imaging scanner, a blip-reversed reference dataset;
acquiring, by the medical imaging scanner, an acquisition dataset of a region of a patient;
estimating, using a deep learning model, a displacement field based on the blip-reversed reference dataset, the deep learning model comprising encoder-decoder architecture with one or more deformable convolutional modulation blocks; and
correcting the acquisition dataset using the displacement field.

16. The method of claim 15, wherein acquiring the blip-reversed reference dataset uses a reference TE that is different from an Acquisition TE used to acquire the acquisition dataset of the patient.

17. The method of claim 15, wherein the medical imaging scanner comprises a magnetic resonance scanner configured to perform EPI DWI.

18. The method of claim 15, wherein the region comprises a region other than a brain of the patient.

19. The method of claim 15, wherein the deep learning model comprises a plurality of resolution layers comprising a Def-ConvFormer block and a down sampling block or up sampling block, wherein a residual connection to an input is made in a last resolution level, wherein a 1×1 convolution is used to estimate the displacement field, wherein a spatial transformer block is used for unwarping and Jacobian modulation for intensity correction of the acquisition dataset using the estimated displacement field.

20. The method of claim 15, wherein the deep learning model is trained at least partially on blip-reversed reference scan data.
